(19) United States
(12) Patent Application Publication
(10) Pub. No.: US 2024/0294444 A1
(43) Pub. Date: Sep. 5, 2024

(54) A METHOD AND FEED FOR PRODUCING ETHYLENE

(71) Applicant: Neste Oyj, Espoo (FI)

(72) Inventors: Otto ROSSI, Porvoo (FI); Andres MUÑOZ GANDARILLAS, Porvoo (FI); John JAMIESON, Porvoo (FI); Antti OJALA, Porvoo (FI)

(73) Assignee: Neste Oyj, Espoo (FI)

(21) Appl. No.: 18/573,544

(22) PCT Filed: Apr. 8, 2022
(86) PCT No.: PCT/FI2022/050230

§ 371 Date: Dec. 22, 2023

(30) Foreign Application Priority Data
Jun. 30, 2021 (FI) 20215772

(51) Int. Cl. C07C 4/04; C07C 11/04

(52) U.S. Cl. CPC C07C 4/04; C07C 11/04

(57) ABSTRACT

Thermal cracking of a feed that contains propane and molecular hydrogen is disclosed. Also, a thermal cracking feed and a thermal cracking effluent are provided.

### TECHNICAL FIELD

The present disclosure generally relates to thermal cracking. The disclosure relates particularly, though not exclusively, to thermal cracking of a propane containing feed derived at least partially from renewable sources.

### BACKGROUND

This section illustrates useful background information without admission of any technique described herein representative of the state of the art.

Ethylene and propylene are commonly used as raw material in petrochemical industry. For example, ethylene and propylene are used to make various chemicals and polymers, such as polyethylene and polypropylene.

Conventionally, ethylene and propylene have been obtained by steam cracking of fossil cuts derived from crude oil and the like, such as fossil ethane, fossil LPG and fossil naphtha.

Recently, steam cracking feeds boiling in the naphtha and diesel ranges derived from renewable sources have been suggested as alternatives that may provide more environmentally sustainable cracking products compared to fossil counterparts. In production of these naphtha and diesel range renewable feeds gaseous side products are formed. Presently, these gaseous side products are primarily burnt as fuel gas.

There is a need to provide further alternatives to the fossil-based steam cracking feeds and processes. Also, there is a need to provide value added use of gaseous side products formed in the production of renewable naphtha and diesel range feeds or products.

### SUMMARY

The appended claims define the scope of protection. Any examples and technical descriptions of apparatuses, products and/or methods in the description and/or drawings not covered by the claims are presented as examples useful for understanding the invention.

According to a first example aspect there is provided a method comprising providing a thermal cracking feed comprising molecular hydrogen (H₂) and from 10 mol-% to 60 mol-% propane based on the total dry amount of substance of the thermal cracking feed, in which thermal cracking feed the ratio of the mol-% amount of propane to the mol-% amount of molecular hydrogen is within a range from 0.10 to 2.5; and subjecting the thermal cracking feed to thermal cracking to obtain a thermal cracking effluent comprising ethylene.

According to a second example aspect there is provided a thermal cracking feed comprising molecular hydrogen (H₂) and from 10 mol-% to 60 mol-% propane based on the total dry amount of substance of the thermal cracking feed, in which thermal cracking feed the ratio of the mol-% amount of propane to the mol-% amount of molecular hydrogen is within a range from 0.10 to 2.5 and the ratio of the mol-% amount of hydrocarbons having a carbon number of at least C2 to the mol-% amount of molecular hydrogen is within a range from 0.10 to 2.5.

According to a third example aspect there is provided a thermal cracking effluent comprising propylene and at least 20 wt-%, preferably at least 25 wt-%, more preferably at least 28 wt-%, even more preferably at least 30 wt-%, such as at least 32 wt-%, ethylene based on the total dry weight of the thermal cracking effluent, in which thermal cracking effluent the ratio of the wt-% amount of propylene to the wt-% amount of ethylene is less than 0.40, preferably less than 0.30, more preferably less than 0.20, such as less than 0.15, and wherein the thermal cracking effluent comprises less than 5.0 wt-%, preferably less than 3.0 wt-%, more preferably less than 2.5 wt-% hydrocarbons having a carbon number of at least C5 based on the total weight of the thermal cracking feed.

According to a fourth example aspect there is provided a thermal cracking effluent obtained or obtainable with the method of the first example aspect.

The present method and thermal cracking feed are advantageous in that they provide high propane conversion, a high specific yield per valuable hydrocarbons of C2 hydrocarbons, good selectivity towards ethylene and a low coking rate. Also, an advantage of the present method and thermal cracking feed is that value added use of gaseous side products from hydrotreatment of renewable oxygen containing hydrocarbons may be provided. Hence, the present method and thermal cracking feed may provide a simple and low-cost method to produce valuable hydrocarbons from renewable oils and fats.

Different non-binding example aspects and embodiments have been illustrated in the foregoing. The embodiments in the foregoing are used merely to explain selected aspects or steps that may be utilized in different implementations. Some embodiments may be presented only with reference to certain example aspects. It should be appreciated that corresponding embodiments may apply to other example aspects as well.

### BRIEF DESCRIPTION OF THE FIGURES

Some example embodiments will be described with reference to the accompanying figures, in which:

FIG. 1 schematically shows an example embodiment of the method of the present disclosure.

### DETAILED DESCRIPTION

In the following description, like reference signs denote like elements or steps.

Unless otherwise mentioned, in the context of the present disclosure, mol-% and wt-% are given based on dry compositions, i.e. a total weight or total amount of substance from which possible H₂O content has been excluded (total weight or total amount of substance without possible H₂O content). Such total weight or total amount of substance from which possible H₂O content has been excluded is referred herein to as dry weight or dry amount of substance, respectively.

In the context of the present disclosure, selectivity towards ethylene refers to the ratio of the wt-% amount of propylene to the wt-% amount of ethylene in a thermal cracking effluent, wherein the wt-% amounts are based on the total dry weight of the thermal cracking effluent (total weight of the thermal cracking effluent excluding the weight of possible H₂O content). A lower ratio of the wt-% amount of propylene to the wt-% amount of ethylene in a thermal cracking effluent means improved selectivity towards ethylene.

In the context of the present disclosure, specific yield per valuable hydrocarbons refers to the wt-% amount of a compound or the sum of the wt-% amounts of certain compounds in a thermal cracking effluent divided by the sum of the wt-% amounts of hydrocarbons having a carbon number of at least C2 (C2+ hydrocarbons) in the thermal cracking feed, wherein the wt-% amounts are based on the total dry weight of the thermal cracking feed and the thermal cracking effluent, respectively. The specific yield per valuable hydrocarbons can be expressed in percentages by multiplying the specific yield per valuable hydrocarbons with 100%.

As used herein C2+ refers to compounds having a carbon number of at least C2 (C2 and higher). C2+ hydrocarbons refer in the context of this disclosure to hydrocarbons having a carbon number of at least C2.

As used herein C4+ refers to compounds having a carbon number of at least C4 (C4 and higher). C4+ hydrocarbons refer in the context of this disclosure to hydrocarbons having a carbon number of at least C4.

As used herein C5+ refers to compounds having a carbon number of at least C5 (C5 and higher). C5+ hydrocarbons refer in the context of this disclosure to hydrocarbons having a carbon number of at least C5.

As used herein C6+ refers to compounds having a carbon number of at least C6 (C6 and higher). C6+ hydrocarbons refer in the context of this disclosure to hydrocarbons having a carbon number of at least C6.

As used herein C10+ refers to compounds having a carbon number of at least C10 (C10 and higher). C10+ hydrocarbons refer in the context of this disclosure to hydrocarbons having a carbon number of at least C10.

Paraffins refer herein to normal paraffins (n-paraffins), isoparaffins (i-paraffins), or both.

Oxygen containing hydrocarbons refer in the context of this disclosure to organic molecules of carbon, hydrogen, and oxygen.

As used herein, the term renewable refers to compounds or compositions that are obtainable, derivable, or originating from plants and/or animals, including materials and products obtainable, derivable, or originating from fungi and/or algae. As used herein, renewable raw material may comprise gene manipulated renewable raw material.

Renewable raw material may also be referred to as biological raw material or biogenic raw material.

As used herein, the term fossil refers to compounds or compositions that are obtainable, derivable, or originating from naturally occurring non-renewable compositions, such as crude oil, petroleum oil/gas, shale oil/gas, natural gas, or coal deposits, and the like, and combinations thereof, including any hydrocarbon-rich deposits that can be utilised from ground/underground sources. The term fossil may also refer to recycling material originating from non-renewable sources.

Said renewable and fossil compounds or compositions are considered differing from one another based on their origin and impact on environmental issues. Therefore, they are treated differently under legislation and regulatory framework.

Typically, renewable and fossil compounds or compositions are differentiated based on their origin and information thereof provided by the producer. However, chemically the renewable or fossil origin of any organic compounds, including hydrocarbons, can be determined e.g. by isotopic carbon distribution involving ¹⁴C, ¹³C and/or ¹²C as described in ASTM D6866:2018. A renewable compound or composition or at least a partly renewable composition is characterised by mandatorily having a higher content of ¹⁴C isotopes than similar components derived from fossil sources. Said higher content of ¹⁴C isotopes is an inherent feature characterising the renewable compound or composition and distinguishing it from fossil compounds and compositions. Thus, in compositions wherein the compositions are based on partly fossil based material and partly renewable component(s), the renewable component can be determined by measuring the ¹⁴C activity. Analysis of ¹⁴C (also referred to as carbon dating or radiocarbon analysis) is an established approach to determine the age of artefacts based on the rate of decay of the isotope ¹⁴C, as compared to ¹²C. This method may be used for determining the physical percentage fraction of renewable materials in bio/fossil mixtures as renewable material is far less aged than fossil material and so the types of material contain very different ratios of ¹⁴C:¹²C. Thus, a particular ratio of said isotopes can be used as a “tag” to identify a renewable carbon compound and differentiate it from non-renewable carbon compounds. While the renewable component reflects the modern atmospheric ¹⁴C activity, very little ¹⁴C is present in fossil material, such as oil, coal and derivatives thereof. Therefore, the renewable fraction of a composition or component is proportional to its ¹⁴C content. Samples of compositions may be analysed to determine the amount of renewable sourced carbon in the composition. This approach would work equally for co-processed compositions or compositions produced from mixed feedstocks. It is to be noted that there is not necessarily any need to test input materials or blending components when using this approach as renewable content of a composition may be directly measured. The isotope ratio does not change in the course of chemical reactions. Therefore, the isotope ratio can be used for identifying renewable compounds, components, and compositions and distinguishing them from non-renewable, fossil materials.

Biological material may have about 100 wt-% renewable (i.e. contemporary or biobased or biogenic) carbon, ¹⁴C, content which may be determined using radiocarbon analysis by the isotopic distribution involving ¹⁴C, ¹³C and/or ¹²C as described for example in ASTM D6866 (2018). Other examples of a suitable method for analysing the content of carbon from biological or renewable origin are DIN 51637 (2014) or EN 16640 (2017).

The term hydrotreatment, sometimes also referred to as hydroprocessing, refers in the context of the present disclosure to a catalytic process of treating organic material by means of molecular hydrogen. Preferably, hydrotreatment removes oxygen from organic oxygen compounds as water i.e. hydrodeoxygenation (HDO), removes sulphur from organic sulphur compounds as dihydrogen sulphide (H₂S), i.e. hydrodesulphurisation (HDS), removes nitrogen from organic nitrogen compounds as ammonia (NH₃), i.e. hydrodenitrogenation (HDN), removes halogens, for example chlorine from organic chloride compounds as hydrochloric acid (HCl), i.e. hydrodechlorination (HDCI), removes metals by hydrodemetallization, and hydrogenates unsaturated bonds present. As used in the context of the present disclosure, hydrotreatment covers or encompasses also hydroisomerisation.

The term hydrodeoxygenation (HDO) refers in the context of this disclosure to removal of oxygen from organic molecules as water by means of molecular hydrogen under the influence of catalyst.

The term deoxygenation refers in the context of this disclosure to removal of oxygen from organic molecules, such as fatty acid derivatives, alcohols, ketones, aldehydes or ethers by any means previously described or by decarboxylation or decarbonylation.

The present disclosure provides a method comprising: providing a thermal cracking feed comprising molecular hydrogen (H₂) and from 10 mol-% to 60 mol-% propane based on the total dry amount of substance of the thermal cracking feed, in which thermal cracking feed the ratio of the mol-% amount of propane to the mol-% amount of molecular hydrogen is within a range from 0.10 to 2.5; and subjecting the thermal cracking feed to thermal cracking to obtain a thermal cracking effluent comprising ethylene.

Surprisingly, it has been found that thermally cracking a feed comprising from 10 mol-% to 60 mol-% propane and molecular hydrogen (H₂) in such amount that the ratio of the mol-% amount of propane to the mol-% amount of molecular hydrogen in the thermal cracking feed is within a range from 0.10 to 2.5 provides good propane conversion and selectivity towards ethylene (a low ratio of the wt-% amount of propylene to the wt-% amount of ethylene in the thermal cracking effluent) and significantly reduces coke formation during the thermal cracking especially compared to thermal cracking of feeds containing higher mol-% of propane and/or without or with lower H₂ content, while providing viable process economy on industrial scale.

Providing a thermal cracking feed comprising from 10 mol-% to 60 mol-% propane based on the dry amount of substance is advantageous in that higher propane contents (above 60 mol-%) in a thermal cracking feed increase the coking rate, decreases propane conversion, and decrease the selectivity towards ethylene, while lower propane contents (less than 10 mol-%) would lead to poor process economy not being sensible on industrial scale. A ratio of the mol-% amount of propane to the mol-% amount of H₂ in the thermal cracking feed within a range from 0.10 to 2.5 was surprisingly found e.g. to significantly reduce the coke formation during thermal cracking compared to thermally cracking feeds in which the ratio of propane to H₂ is higher, and for example compared to feeds with no H₂ content. Also, a lower propane to H₂ ratio would not provide for sensible process economy on an industrial scale. Processing high volumes of feed not comprising sufficiently valuable hydrocarbons (C2+ hydrocarbons, such as propane) affects the overall process economy, as well as reduces production of the desired products (such as ethylene, propylene). Furthermore, thermal cracking feeds having higher H₂ content are more difficult to transport between production facilities because liquefying such feeds by compression would not be feasible on industrial scale, or at least not sensible in terms of process economy.

Further advantages of providing a thermal cracking feed containing from 10 mol-% to 60 mol-% propane and H₂ in such amount that the ratio of the mol-% amount of propane to the mol-% amount of H₂ in the thermal cracking feed is within a range from 0.10 to 2.5 include, compared to feeds having a propane and/or H₂ content outside said ranges: lower yields of butadiene, pyrolysis gasoline (C5-C9 hydrocarbons), acetylene, aromatics, especially BTX (benzene, toluene, xylene), particularly benzene, MAPD contaminants (methyl acetylene and propadiene), C10+ compounds, and a higher ratio of the wt-% amount of propylene to the wt-% amount of all C3 compounds in the thermal cracking effluent. A higher ratio of the wt-% amount of propylene to the wt-% amount of all C3 compounds in the thermal cracking effluent is beneficial in that it facilitates separation (purification) of propylene from the other close-boiling C3 compounds, especially propane. Also, the energy needed for propylene purification is reduced. A lower yield of benzene is beneficial as benzene typically needs to be removed from C5-C9 hydrocarbons usable as fuel component(s) due to strict benzene limits in traffic fuels. Lower yields of C5-C9 hydrocarbons and C10+ compounds contribute to the reduction of the coke formation. The present method also provides a high specific yield per valuable hydrocarbons of C2+C3 hydrocarbons (sum of specific yields per valuable hydrocarbons of C2 hydrocarbons and of C3 hydrocarbons), particularly of C2 hydrocarbons. This is beneficial because ethylene and propylene are valuable and desired products and ethane and propane can optionally be recycled back to the steam cracking process to produce more valuable hydrocarbons. Optionally, propane can be separated and e.g. converted into valuable chemicals by on-purpose technologies.

It has surprisingly been found that H₂ in the thermal cracking feed does not merely dilute the hydrocarbon content in the thermal cracking feed, but also impacts the chemistry during thermal cracking. Hence, advantages of the thermal cracking feed of the present disclosure are not obtained due to propane content (or content of C2+ hydrocarbons) alone, but the presence and content of H_{z} are also important. Without being bound to any theory, it is believed that H₂ in the thermal cracking feed controls formation and/or further reactions of reactive species like unsaturated compounds, although the underlying mechanism is not known. It is also surprising that despite of the presence of H₂ in the thermal cracking feed unsaturated compounds, especially ethylene and also propylene, are obtained with good yields, i.e. the presence of H₂ does not lead to saturation of the double bonds of these.

Although H₂ may sometimes be produced in thermal cracking, for example in amounts from about 1 wt-% to less than 2 wt-% of the thermal cracking effluent, H₂ formed during thermal cracking alone is not sufficient for obtaining the benefits of having H₂ present in the thermal cracking feed already from the beginning of the thermal cracking. Without being bound to any theory and although the underlying mechanism is not known, it is believed that the early presence of H₂ in the thermal cracking prevents or reduces formation of highly reactive species, or quenches them, thereby controlling the chain of subsequent reactions. Controlling of said chain of subsequent reaction may, without being bound to any theory, cause the relatively low amount of C10+ compounds in the steam cracking effluent.

A main benefit of thermal cracking of the feed of the present disclosure is less coke formation, i.e. reduction of the coking rate. Coking is an undesired side reaction in thermal cracking, such as steam cracking, and a major operational problem in the thermal cracking equipment, e.g. steam cracking equipment, especially in the radiant section of steam cracker furnaces and transfer line exchangers. Coke may be formed in different ways and forms, e.g. filamentous coke may be formed by surface catalysed reactions, for example caused by nickel and iron on equipment's alloy surfaces, and amorphous coke may be formed in the gas phase.

Coke formation may cause high production losses due to increased pressure drop, impaired heat transfer and higher feed consumption since part of the feed's carbon content is lost as the formed coke. Coke formation may lead to continuous temperature increase of external tube surfaces thereby influencing process selectivity and increasing coke formation rate even further. Reducing the coke formation alleviates these problems.

The formed coke may be removed in decoking cycles by controlled combustion for example with steam and air. However, this causes production losses due to non-productive downtime as the decoking cycle cannot, for a certain equipment, be performed simultaneously with thermal cracking. Decoking cycles can also cause wear on the equipment and shorten the thermal cracker furnaces' coil life. A decreased coking rate prolongs the time between decoking cycles (reduces downtime of the equipment) and allows for less frequent decoking cycles thus reducing wear on the equipment.

Preferably, the thermal cracking feed of the present disclosure comprises propane from 15 mol-% to 50 mol-%, further preferably from 20 mol-% to 45 mol-%, more preferably from 20 mol-% to 40 mol-%, even more preferably from 20 mol-% to 35 mol-%, such as from 20 mol-% to 30 mol-%, based on the total dry amount of substance of the thermal cracking feed. Such amounts of propane in the thermal cracking feed provides good propane conversion, good selectivity towards ethylene, contribute to a low coking rate and good process economy.

Preferably, in the thermal cracking feed of the present disclosure the ratio of the mol-% amount of propane to the mol-% amount of molecular hydrogen is within a range from 0.10 to 2.2, more preferably from 0.18 to 2.2, and even more preferably from 0.18 to 2.0 based on the total dry amount of substance of the thermal cracking feed. Such propane to H₂ ratios improve process economy while controlling the coking rate. A propane to H₂ ratio at or slightly above the lower limits enhances coke control (reduces coking rate) whereas a propane to H₂ ratio at or slightly below the upper limits improves process economy. The preferred ranges provide desirable balance between controlling (reducing) the coking rate and ensuring desirable process economy.

In certain embodiments, the thermal cracking feed comprises from 5 mol-% to 80 mol-% H₂ based on the total dry amount of substance of the thermal cracking feed. Such mol-% H₂ in the thermal cracking feed decreases the coking rate compared to feeds with no or lower H₂ content while still providing sensible process economy on industrial scale. It was found that within said range, the higher the H₂ content of the thermal cracking feed, the lower the coking rate, and the lower the H₂ content of the thermal cracking feed, the better the overall process economy. Preferably, the thermal cracking feed comprises H₂ from 10 mol-% to 80 mol-%, more preferably from 20 mol-% to 75 mol-%, and even more preferably from 30 mol-% to 70 mol-% based on the total dry amount of substance of the thermal cracking feed. The preferred ranges provide a balance between decreasing the coking rate and providing good process economy.

In certain embodiments, the thermal cracking feed comprises from 0 mol-% to 10 mol-%, preferably from 0 mol-% to 6 mol-%, more preferably from 0 mol-% to 4 mol-% ethane based on the total dry amount of substance of the thermal cracking feed. It was surprisingly found that the present method favours ethylene formation even when the thermal cracking feed is without ethane content or merely comprises low amounts of ethane.

In certain embodiments, the thermal cracking feed comprises from 0 mol-% to 8 mol-%, preferably from 1 mol-% to 8 mol-%, more preferably from 2 mol-% to 8 mol-% hydrocarbons having a carbon number of at least C4 based on the total dry amount of substance of the thermal cracking feed. A C4+ hydrocarbon content of 8 mol-% or less provides a more uniform thermal cracking feed for which thermal cracking conditions are easier to optimise compared to similar feeds with higher amount of C4+ hydrocarbons. Also, the risk of condensation of C4+ hydrocarbons in the thermal cracking process, increasing for example coke formation, is lower compared to thermal cracking feeds with a higher C4+ content. However, presence of some C4+ hydrocarbons is beneficial for increasing the ethylene yield in the thermal cracking. Ethylene production in the thermal cracking is enhanced when the thermal cracking feed contains some C4+ hydrocarbons compared for example to thermal cracking feeds of pure propane.

In thermal cracking, it is important that the thermal cracking feed is vaporised and maintained in gaseous phase during the thermal cracking process. C5+ and C6+ compounds are prone to condensate (compared to lighter species), which may cause problems, such as increased coke formation. It is hence beneficial to control the amount of C5+ and C6+ compounds in the thermal cracking feed. Typically, reducing the amount of C6+ hydrocarbons is easier compared to reducing the amount of C5 hydrocarbons.

Preferably, the thermal cracking feed of the present disclosure comprises hydrocarbons having a carbon number of at least C5 (C5+ hydrocarbons) from 0 mol-% to 8 mol-%, preferably from 0 mol-% to 6 mol-%, more preferably from 0 mol-% to 4 mol-% based on the total dry amount of substance of the thermal cracking feed. This lowers the risk of condensation of portions of the feed during the thermal cracking and provides a more uniform thermal cracking feed composition facilitating optimisation of process conditions.

In certain embodiments, the ratio of the mol-% amount of hydrocarbons having a carbon number of at least C2 (based on the total dry amount of substance of the thermal cracking feed) to the mol-% amount of molecular hydrogen (based on the total dry amount of substance of the thermal cracking feed) in the thermal cracking feed is within a range from 0.10 to 2.5, preferably within a range from 0.10 to 2.2, more preferably within a range from 0.18 to 2.2, even more preferably within a range from 0.18 to 2.0. Total content of hydrocarbons having a carbon number of at least C2 (comprising propane, optional ethane, and optional C4+ hydrocarbons) in the thermal cracking feed can be regarded as the total content of valuable hydrocarbons in the feed, because it is from these species the desired ethylene (and propylene) are formed in the thermal cracking. Said ranges of the ratio of the mol-% amount of hydrocarbons having a carbon number of at least C2 to the wt-% amount of H₂ are beneficial in that they provide enough H₂ to interact with the C2+ hydrocarbons as they will crack to olefins while still providing for viable process economy.

The thermal cracking feed may comprise gaseous impurities like methane, CO, CO₂, NH₃, and H₂S. In certain embodiments, the sum of the mol-% amounts of methane, CO, CO₂, NH₃, and H₂S in the thermal cracking feed is within a range from 0 mol-% to 15 mol-% or from 0.1 mol-% to 15 mol-%, preferably from 0 mol-% to 10 mol-% or from 0.1 mol-% to 10 mol, more preferably from 0 mol-% to 8 mol-% or from 0.1 mol-% to 8 mol-% based on the total dry amount of substance of the thermal cracking feed. It was surprisingly found that presence of methane, CO, CO₂, NH₃, and H₂S in the thermal cracking feed is less harmful to the thermal cracking process and the product distribution of the thermal cracking effluent than expected. In fact, it was found that the method of the present disclosure performs well even if the sum of the mol-% amounts of methane, CO, CO₂, NH₃, and H₂S is up to 15 mol-% of the thermal cracking feed. This is beneficial since purification of these impurities may be avoided or be less extensive. Also, it was surprisingly found that conversion of valuable hydrocarbons (C2+ hydrocarbons) in the thermal cracking feed to target products (such as ethylene and propylene) is increased due to dilution effect of at least CO₂ and methane.

In certain embodiments, the thermal cracking feed comprises CO from 0 mol-% to 2 mol-%, preferably from 0.2 mol-% to 1.8 mol-% based on the total dry amount of substance of the thermal cracking feed. The method of the present invention tolerates such amounts of CO surprisingly well, and the need of CO purification may be reduced or may be omitted. The CO in the thermal cracking feed is typically carried over to the thermal cracking effluent and hence a low amount of CO in the thermal cracking feed may also reduce or omit the need to purify CO from thermal cracking products. A low amount of CO in thermal cracking products is desirable particularly if the products are used as starting material in polymerisation processes as CO is a polymerisation catalyst poison.

Preferably, in the thermal cracking feed of the present disclosure the sum of the mol-% amounts of hydrocarbons having a carbon number of at least C2 (C2+ hydrocarbons) and of molecular hydrogen (H₂) is at least 85 mol-%, more preferably at least 90 mol-%, even more preferably at least 92 mol-% based on the total dry amount of substance of the thermal cracking feed. Such thermal cracking feeds provide a beneficial thermal cracking product distribution and a low coking rate.

In certain particularly preferred embodiments, the sum of the mol-% amounts of hydrocarbons having carbon number C2 (C2 hydrocarbons), hydrocarbons having carbon number C3 (C3 hydrocarbons) and molecular hydrogen (H₂) in the thermal cracking feed is at least 85 mol-%, preferably at least 90 mol-%, more preferably at least 92 mol-% based on the total dry amount of substance of the thermal cracking feed. Such thermal cracking feeds provide a particularly beneficial thermal cracking product distribution, a low coking rate, and facilitates optimisation of process conditions due to the uniform composition of the thermal cracking feed.

In certain preferred embodiments, the thermal cracking feed comprises, based on the total dry amount of substance of the thermal cracking feed, from 10 mol-% to 60 mol-% propane, from 0 mol-% to 10 mol-% ethane, and from 0 mol-% to 8 mol-% hydrocarbons having a carbon number of at least C4, in which thermal cracking feed the molar ratio of the mol-% amount of propane to the mol-% amount of molecular hydrogen is within a range from 0.10 to 2.5, the ratio of the mol-% amount of hydrocarbons having a carbon number of at least C2 to the mol-% amount of molecular hydrogen in the thermal cracking feed is within a range from 0.10 to 2.5, and the sum of the mol-% amounts of methane, CO, CO₂, NH₃, and H₂S is within a range from 0 mol-% to 15 mol-%.

Conversion from mol-% to wt-% or from wt-% to mol-% cannot be done for single components in isolation but requires consideration of the composition as a whole. For example, the amount of heavier compounds, such as C4+ hydrocarbons, in a composition (thermal cracking feed) significantly affects conversion from mol-% to wt-% or from wt-% to mol-%.

In certain embodiments, the thermal cracking feed comprises from 39.5 wt-% to 97 wt-% propane based on the total dry weight of the thermal cracking feed. In certain embodiments, the thermal cracking feed comprises from 2 wt-% to 15 wt-% H₂ based on the total dry weight of the thermal cracking feed.

By way of example, the thermal cracking feed of the present disclosure may comprise, based on the total dry weight of the thermal cracking feed, from 39.5 wt-% to 97 wt-% propane, from 2 wt-% to 15 wt-% H₂, from 0 wt-% to 18 wt-%, such as from 0 wt-% to 10 wt-%, ethane, from 0 wt-% to 37 wt-%, such as from 0 wt-% to 15 wt-%, hydrocarbons having a carbon number of at least C4, in which thermal cracking feed the sum of the wt-% amounts of CO, CO₂, NH₃ and H₂S is within a range 0 wt-% from 8 wt-%.

Preferably, in the context of the present disclosure, the thermal cracking feed is a renewable or partially renewable thermal cracking feed wherein the biogenic carbon content is at least 50 wt-%, preferably at least 70 wt-%, more preferably at least 90 wt-%, even more preferably about 100 wt-% based on the total weight of carbon (TC) of the thermal cracking feed (EN 16640 (2017)). A renewable thermal cracking feed may be regarded environmentally more sustainable compared to fossil feeds. A renewable or partially renewable thermal cracking feed yields a renewable or partially thermal cracking effluent, respectively, which renewable or partially renewable thermal cracking feed may then be further processed into renewable or partially renewable compositions, compounds, and other products, typically each being regarded environmentally more sustainable compared to fossil counterparts. Preferably, the biogenic carbon content of the thermal cracking effluent is at least 50 wt-%, preferably at least 70 wt-%, more preferably at least 90 wt-%, even more preferably about 100 wt-% based on the total weight of carbon (TC) of the thermal cracking effluent (EN 16640 (2017)).

In certain embodiments, at least a portion of the thermal cracking feed is obtained as a gaseous side product of hydrotreatment of renewable oxygen containing hydrocarbons, optionally after having subjected said gaseous side product to purification treatment. As used herein, said gaseous side product refers to a composition of compounds that are gaseous at NTP (normal temperature and pressure, i.e. 20° C. and an absolute pressure of 1 atm (101.325 kPa)) and water.

In certain embodiments, the thermal cracking feed is a co-feed of a gaseous side product of hydrotreatment of renewable oxygen containing hydrocarbons, optionally after having subjected said gaseous side product to purification, and commercially available fossil gases consisting essentially of propane and/or butane, such as fossil LPG, or any hydrocarbon containing gas stream(s) from a conventional fossil refinery, said steams(s) preferably comprising at least 50 wt-% C2-C4 hydrocarbons, such as gaseous effluent (after optional purification) from fluid catalytic cracking (FCC) or fossil oil refinery hydrotreatment.

Preferably, the gaseous side product of hydrotreatment of renewable oxygen containing hydrocarbons is gaseous fraction from gas-liquid separation of hydrotreatment effluent from hydrotreatment of renewable oxygen containing hydrocarbons, such as renewable oils and/or fats. As used herein, gaseous fraction includes or consists essentially of compounds that are gaseous at NTP (normal temperature and pressure) and water.

Preferably, the thermal cracking feed of the present disclosure is obtainable or obtained as gaseous fraction from gas-liquid separation of hydrotreatment effluent from hydrotreatment of renewable oxygen containing hydrocarbons, such as renewable oils and/or fats, which gaseous fraction has at least partially been subjected to purification treatment. Preferably, the purification treatment comprises at least separation of molecular hydrogen (H₂) from the gaseous fraction from gas-liquid separation of hydrotreatment effluent. The separated H₂ may be recovered and recycled back to the hydrotreatment of renewable oxygen containing hydrocarbons. Separating and recycling some of the H₂ contained in the gaseous fraction improves process economy. Other purification treatments may include removal of sulfur containing compounds, such as H₂S, and optionally CO₂ for example by amine wash (amine scrubber).

In certain embodiments, providing a thermal cracking feed comprises subjecting renewable oxygen containing hydrocarbons to hydrotreatment comprising deoxygenation and optionally isomerisation to obtain hydrotreatment effluent, which renewable oxygen containing hydrocarbons preferably comprises one or more of fatty acids, fatty acid esters, resin acids, resin acid esters, sterols, fatty alcohols, oxygenated terpenes, and other renewable organic acids, ketones, alcohols, and anhydrides, separating gaseous fraction from the hydrotreatment effluent, and providing the gaseous fraction optionally after having subjected at least a portion of it to purification treatment, optionally mixed with a gaseous fossil co-feed, such as fossil LPG, as the thermal cracking feed. Preferably, the isomerisation is hydroisomerisation. Preferably, the hydrotreatment is catalytic hydrotreatment comprising HDO. The gaseous fossil co-feed may be commercially available fossil gases consisting essentially of propane and/or butane, such as fossil LPG, or any hydrocarbon containing gas stream(s) from a conventional fossil refinery, said steams(s) preferably comprising at least 50 wt-% C2-C4 hydrocarbons, such as gaseous effluent (after optional purification) from FCC or fossil oil refinery hydrotreatment.

An advantage of the method of the present disclosure is that it may provide value added use of gaseous side products from hydrotreatment of renewable oxygen containing hydrocarbons. Conventionally, these gaseous side products have, optionally after separation of recycling streams, been combusted. The composition of the gaseous fraction from gas-liquid separation of hydrotreatment effluent from hydrotreatment of renewable oxygen containing hydrocarbons remains rather constant regardless of whether the hydrotreatment process is adjusted to produce as main product naphtha, diesel, or aviation range paraffins and regardless of the desired isomerisation degree thereof. Hence, the present method may provide value added use of gaseous side products from a broad range of hydrotreatment processes of renewable oxygen containing hydrocarbons. Also, the present method does not limit flexibility to adjust the hydrotreatment process to meet changing market demand of the various paraffin fractions.

Renewable oxygen containing hydrocarbons may also be referred to as biological oxygen containing hydrocarbons, bio-based oxygen containing hydrocarbons, or biogenic oxygen containing hydrocarbons. Preferably, the biogenic carbon content of the renewable oxygen containing hydrocarbons is at least 90 wt-%, more preferably at least 95 wt-%, even more preferably about 100 wt-% based on the total weight of carbon (TC) in the renewable oxygen containing hydrocarbons (EN 16640 (2017)). Typically, organic compounds derived from fossil sources, such as crude oil based mineral oil, have a biogenic carbon content of about 0 wt-%.

Most renewable raw material comprises materials having a high oxygen content. The renewable oxygen containing hydrocarbons may include one or more of fatty acids, whether in free or salt form; fatty acid esters, such as mono-, di- and triglycerides, alkyl esters such as methyl or ethyl esters, etc; resin acids, whether in free or salt form; resin acid esters, such as alkyl esters, sterol esters etc; sterols; fatty alcohols; oxygenated terpenes; and other renewable organic acids, ketones, alcohols, and anhydrides.

Preferably, the renewable oxygen containing hydrocarbons originate or are derived from one or more of vegetable oils, such as rapeseed oil, canola oil, soybean oil, coconut oil, sunflower oil, palm oil, palm kernel oil, peanut oil, linseed oil, sesame oil, maize oil, poppy seed oil, cottonseed oil, soy oil, tall oil, corn oil, castor oil, jatropha oil, jojoba oil, olive oil, flaxseed oil, camelina oil, safflower oil, babassu oil, seed oil of any of Brassica species or subspecies, such as Brassica carinata seed oil, Brassica juncea seed oil, Brassica oleracea seed oil, Brassica nigra seed oil, Brassica napus seed oil, Brassica rapa seed oil, Brassica hirta seed oil and Brassica alba seed oil, and rice bran oil, or fractions or residues of said vegetable oils such as palm olein, palm stearin, palm fatty acid distillate (PFAD), purified tall oil, tall oil fatty acids, tall oil resin acids, distilled tall oil, tall oil unsaponifiables, tall oil pitch (TOP), and used cooking oil preferably of vegetable origin; animal fats, such as tallow, lard, yellow grease, brown grease, fish fat, poultry fat, and used cooking oil of animal origin; microbial oils, such as algal lipids, fungal lipids and bacterial lipids.

Optionally, the vegetable oils, animal fats and/or microbial oils from which the oxygen containing hydrocarbons originate or are derived may have been subjected to a pre-treatment for example to remove impurities, preferably S, N and/or P and/or metal-containing impurities, from the said oils and/or fats. In certain embodiments, the pre-treatment comprises one or more of washing, degumming, bleaching, distillation, fractionation, rendering, heat treatment, evaporation, filtering, adsorption, hydrodeoxygenation, centrifugation, precipitation, hydrolysis/transesterification of glycerides, and/or partial or full hydrogenation.

Renewable oxygen containing hydrocarbons originating from renewable oils and/or fats typically comprise C10-C24 fatty acids and derivatives thereof, including esters of fatty acids, glycerides, i.e. glycerol esters of fatty acids. The glycerides may specifically include monoglycerides, diglycerides and triglycerides. Optionally, the renewable oxygen containing hydrocarbons may be at least partially derived or obtained from recyclable waste and/or recyclable residue, such as used cooking oil, free fatty acids, palm oil by-products or process side streams, sludge, side streams from vegetable oil processing, or a combination thereof.

Compared to gaseous stream(s) separated from fossil oil refinery hydrotreatment effluent, the gaseous fraction from gas-liquid separation of hydrotreatment effluent from hydrotreatment of renewable oxygen containing hydrocarbons typically contains more CO₂, less aromatics, more CO, more propane, and more H₂O.

Propane and H₂ are typically present in the hydrotreatment effluent from hydrotreatment of renewable oxygen containing hydrocarbons and end up in the gaseous fraction in the gas-liquid separation. Propane and H₂ may be the main components of the gaseous fraction. Propane in the hydrotreatment effluent originates mainly from glycerol backbones of triglyceride containing fatty feedstocks, but some propane may also form via cracking reactions occurring in the hydrotreatment. H₂ is carried over to the hydrotreatment effluent as unreacted hydrotreatment reagent.

Other species of the hydrotreatment effluent that typically end up in the gaseous fraction in the gas-liquid separation include ethane, gaseous impurities, such as methane, CO, CO₂, NH₃ and H₂S, and relatively small amounts of C4+ hydrocarbons, mainly C4-C6 hydrocarbons, especially butane. Also H₂O, originating mainly from hydrodeoxygenation reactions often occurring in the hydrotreatment of renewable oxygen containing compounds, may end up in the gaseous fraction, or H₂O may be removed in the gas-liquid separation. Also NH₃ may be removed in the gas-liquid separation.

The gaseous fraction of the hydrotreatment effluent may contain up to 10 wt-% ethane. Presence of ethane in higher amounts may be a sign of excessive, undesired overcracking during the hydrotreatment.

Species like methane, CO, CO₂, NH₃ and H₂S are typical impurities in the gaseous fraction (gaseous impurities) of the hydrotreatment effluent. CO and CO₂ originate mainly from decarboxylation/decarbonylation reactions, NH₃ from denitrogenation reactions, and H₂S from hydrodesulphurisation reactions during the hydrotreatment of renewable oxygen containing hydrocarbons, such as fatty feedstocks. Methane may be generated in the hydrotreatment by cracking reactions, which cracking reactions may occur not just during a possible hydrocracking step but also in connection with hydrodeoxygenation and hydroisomerisation and similar hydrotreatment steps not as such aiming at cracking.

An advantage of the method of the present disclosure is that these species (methane, CO, CO₂, NH₃ and H₂S) typically regarded as impurities may not need to be removed from the gaseous fraction of the hydrotreatment effluent in order to use the gaseous fraction as thermal cracking feed in accordance with the present disclosure. The present method tolerates these impurities (methane, CO, CO₂, NH₃ and H₂S) to up to 15 mol-% of the total dry amount of substance of the thermal cracking feed. Surprisingly, the presence of said impurities in the thermal cracking feed is even beneficial in that at least CO₂ and methane may dilute the content of propane in the thermal cracking feed thus improving the propane conversion during the thermal cracking.

The hydrotreatment to which the oxygen containing hydrocarbons are subjected may in the context of this disclosure comprise deoxygenation and/or isomerisation reactions of renewable oxygen containing hydrocarbons. Preferably, the hydrotreatment comprises at least deoxygenation reactions of renewable oxygen containing hydrocarbons, preferably at least hydrodeoxygenation.

Hydrotreatment of the oxygen containing hydrocarbons may involve various reactions where molecular hydrogen reacts with other components, or components undergo molecular conversions in presence of molecular hydrogen and a catalyst. The reactions may include but are not limited to hydrogenation, hydrodeoxygenation, hydrodesulphurization, hydrodenitrogenation, hydrodemetallization, hydrocracking, hydropolishing, hydroisomerisation and hydrodearomatization.

Deoxygenation refers herein to removal of oxygen as H₂O, CO₂ and/or CO from the oxygen containing hydrocarbons by hydrodeoxygenation, decarboxylation and/or decarbonylation. Preferably, the hydrotreatment comprises deoxygenation by hydrodeoxygenation (HDO) reactions and optionally isomerisation by hydroisomerisation reactions. Hydrodeoxygenation refers herein to removal of oxygen as H₂O from oxygen containing hydrocarbons by means of molecular hydrogen under influence of a catalyst to obtain hydrocarbons, while hydroisomerisation means formation of branches to hydrocarbons by means of molecular hydrogen under influence of a catalyst that can be same or different as for HDO.

In embodiments, wherein the hydrotreatment comprises deoxygenation and isomerisation, the deoxygenation reactions and the isomerisation reactions may be conducted in a single reactor conducting deoxygenation and isomerisation reactions in same or subsequent catalyst beds, or in separate reactors. Preferably, the deoxygenation and isomerisation reactions of the hydrotreatment are conducted in separate deoxygenation and isomerisation steps in subsequent catalyst beds in same reactor or in separate reactors, preferably in separate reactors.

Reaction conditions and catalysts suitable for the hydrodeoxygenation and isomerisation of renewable oxygen containing hydrocarbons, such as fatty acids and/or fatty acid derivatives, are known. Examples of such processes are presented in WO 2015/101837 A2, paragraphs [0032]-[0037], F1100248, Examples 1-3, EP 1741768 A1, paragraphs [0038]-[0070], particularly paragraphs [0056]-[0070], and Examples 1-6, and EP 2141217 A1, paragraphs [0055]-[0093], particularly paragraphs [0071]-[0093] and Example 1. Also other methods may be employed, particularly another BTL (Biomass-To-Liquid) method may be chosen.

The hydrodeoxygenation of renewable oxygen containing hydrocarbons is preferably performed at a pressure (total pressure) selected from a range from 1 MPa to 20 MPa, preferably from 1 MPa to 15 MPa, more preferably from 3 MPa to 10 MPa, and at a temperature selected from a range from 200 to 500° C., preferably from 280 to 400° C., and optionally at a feed rate (liquid hourly space velocity) selected from a range from 0.1 to 10 h⁻¹ (v/v).

The hydrodeoxygenation may be performed in the presence of known hydrodeoxygenation catalyst containing metal(s) from Group VIII and/or Group VIB of the Periodic System. The catalyst may be supported on any suitable support, such as alumina, silica, zirconia, titania, amorphous carbon, molecular sieve(s), or combinations thereof. Preferably, the hydrodeoxygenation catalyst is supported Pd, Pt, Ni, or NiW catalyst, or supported Mo containing catalyst, such as NiMo or CoMo, catalyst, wherein the support is alumina and/or silica, or a combination of these catalysts. Typically, NiMo/Al₂O₃ and/or CoMo/Al₂O₃ catalysts are used. The hydrodeoxygenation (HDO) of renewable oxygen containing hydrocarbons is preferably carried out in the presence of sulphided NiMo or sulphided CoMo catalysts in the presence of hydrogen gas (H₂). The HDO may be performed under a hydrogen pressure selected from a range from 1 MPa to 20 MPa, at temperatures selected from a range from 200° C. to 400° C., and liquid hourly space velocities selected from a range from 0.2 h⁻¹ to 10 h⁻¹ (v/v).

Using a sulfided catalyst, the sulfided state of the catalyst may be maintained during the HDO step by the addition of sulfur in the gas phase or by using a feedstock having sulphur containing mineral oil blended with the renewable oxygen containing hydrocarbons. The sulfur content of the total feedstock being subjected to hydrodeoxygenation may be, for example, within a range from 50 wppm (ppm by weight) to 20 000 wppm, preferably within a range from 100 wppm to 1000 wppm.

Effective conditions for hydrodeoxygenation may reduce the oxygen content of the renewable oxygen containing hydrocarbons, such as fatty acids or fatty acid derivatives, to less than 20 wt-%, such as less than 0.5 wt-% or less than 0.2 wt-%.

The optional isomerisation is not particularly limited and any suitable approach resulting in isomerisation reactions may be used. However, catalytic hydroisomerisation treatments are preferred. The isomerisation treatment is preferably performed at a temperature selected from a range from 200° C. to 500° C., preferably from 280° C. to 400° C., such as from 300° C. to 350° C., and at a pressure (total pressure) selected from a range from 1 MPa to 15 MPa, preferably from 3 MPa to 10 MPa.

The isomerisation treatment may be performed in the presence of known isomerisation catalysts, for example, catalysts containing a molecular sieve and/or a metal selected from Group VIII of the Periodic System and a support. Preferably, the isomerisation catalyst is a catalyst containing SAPO-11 or SAPO-41 or ZSM-22 or ZSM-23 or ferrierite and Pt, Pd, or Ni and Al₂O₃ or SiO₂. Typical isomerisation catalysts are, for example, Pt/SAPO-11/Al₂O₃, Pt/ZSM-22/Al₂O₃, Pt/ZSM-23/Al₂O₃ and/or Pt/SAPO-11/SiO₂. The catalysts may be used alone or in combination. Catalyst deactivation during the isomerisation treatment may be reduced by the presence of molecular hydrogen in the isomerisation treatment. In certain preferred embodiments, the isomerisation catalyst is a noble metal bifunctional catalyst, such as Pt-SAPO and/or Pt-ZSM catalyst, which is used in combination with molecular hydrogen.

The isomerisation reactions serve to isomerise at least part of the n-paraffins obtained through deoxygenation of renewable oxygen containing hydrocarbons. The isomerisation may comprise intermediate steps such as a purification step and/or a fractionation step. The deoxygenation and isomerisation reactions may be performed either simultaneously or in sequence.

In certain embodiments, hydrotreatment of renewable oxygen containing hydrocarbons comprises subjecting the renewable oxygen containing hydrocarbons to hydrodeoxygenation and hydroisomerisation reactions in a single step on the same catalyst bed using a single catalyst for this combined step, e.g. NiW, or a Pt catalyst, such as Pt/SAPO in a mixture with a Mo catalyst on a support, e.g. NiMo on alumina.

In embodiments wherein the hydrotreatment comprises deoxygenation and isomerisation and wherein deoxygenation and isomerisation are performed in sequence, the deoxygenation is followed by the isomerisation.

After subjecting oxygen containing hydrocarbons to hydrotreatment, the hydrotreatment effluent is fractionated into gaseous fraction and liquid fraction. Separating gaseous fraction from the hydrotreatment effluent may comprise or consist essentially of separating gaseous compounds (gaseous at NTP) and water from the hydrotreatment effluent. Gaseous compounds (NTP) refer herein to compounds that are in gas form under normal temperature and pressure, i.e. 20° C. and an absolute pressure of 1 atm (101.325 kPa).

In certain embodiments, separating gaseous fraction from the hydrotreatment effluent is conducted by subjecting the hydrotreatment effluent to gas-liquid separation. The gas-liquid separation may be conducted as a separate step (e.g. after the hydrotreatment product has left the hydrotreatment reactor or reaction zone) and/or as an integral step of the hydrotreatment step, e.g. within the hydrotreatment reactor or reaction zone. Majority of water contained in the hydrotreatment effluent, formed e.g. during hydrodeoxygenation of renewable oxygen containing hydrocarbons, may be removed from the hydrotreatment effluent in the gas-liquid separation step for example via a water boot.

In certain embodiments, the gas-liquid separation is carried out at a temperature selected from a range from 0° C. to 500° C., such as from 15° C. to 300° C., or from 15° C. to 150° C., preferably from 15° C. to 65° C., such as from 20° C. to 60° C., and preferably at the same pressure as the hydrotreatment. In general, the pressure in the gas-liquid separation step may be within a range from 0.1 to 20 MPa, preferably from 1 to 10 MPa, or from 3 to 7 MPa.

Preferably, at least a portion of the gaseous fraction of the hydrotreatment effluent is subjected to a purification treatment. Preferably, the purification treatment comprises at least separation of molecular hydrogen (H₂) from the gaseous fraction. The purification treatment may also include removal of sulfur containing compounds, preferably H₂S, and optionally CO₂.

In certain embodiments, the purification treatment comprises subjecting at least a portion of the gaseous fraction to purification treatment to remove at least H₂S and optionally CO₂ to obtain a H₂S and optionally CO₂ depleted gaseous stream, and subjecting the H₂S and optionally CO₂ depleted gaseous stream to H₂ separation and optionally to drying. The H₂S and optionally CO₂ depleted gaseous stream contains less H₂S and optionally less CO₂ than the portion of the gaseous fraction subjected to the purification treatment to remove at least H₂S and optionally CO₂. That is, at least some, but not necessary all, H₂S and optionally CO₂ is removed in purification treatment to remove at least H₂S and optionally CO₂.

Preferably, the purification treatment to remove at least H₂S is or comprises amine scrubbing. The H₂S depleted gaseous stream may in certain embodiments comprise H₂S at most 50 ppm by weight, preferably at most 10 ppm by weight, more preferably at most 5 ppm by weight, even more preferably at most 1 ppm by weight. In case CO₂ is removed from the gaseous fraction or a portion thereof, the CO₂ depleted gaseous stream may comprise CO₂ at most 50 000 ppm by weight, preferably at most 5 000 ppm by weight, more preferably at most 500 ppm by weight, even more preferably at most 100 ppm by weight. For example, amine scrubbing may remove CO₂ (in addition to H₂S) from the gaseous fraction. In embodiments, wherein subjecting at least a portion of the gaseous fraction to purification treatment comprises removal of H₂S and optionally CO₂, this step is performed before the H₂ separation.

Separation of molecular H₂ from the gaseous fraction preferably comprises separating H₂ from at least a portion of the gaseous fraction using a membrane separation technique, preferably selective membrane separation. However, other methods for separating H₂ (and optionally at the same time other gaseous components) may be accomplished using any other suitable method, such as cryogenic distillation or swing adsorption.

At hydrotreatment plants of renewable oxygen containing hydrocarbons it is generally desired to recover most of the H₂ from the gaseous fraction of the hydrotreatment effluent and recycle the recovered H₂ back to the hydrotreatment. The gaseous fraction may comprise H₂ from 5 mol-% to 80 mol-%, preferably from 10 mol-% to 80 mol-%, more preferably from 20 mol-% to 75 mol-%, even more preferably from 30 mol-% to 70 mol-% based on the total dry amount of substance of the gaseous fraction. For example, the gaseous fraction may comprise H₂ from 2 wt-% to 15 wt-% based on the total dry weight of the gaseous fraction.

The membrane employed in the membrane separation process is preferably hydrogen selective, in that it selectively permeates H₂. The membrane has a feed side and a permeate side. H₂ rich gas is recovered as permeate.

Various hydrogen permeable membranes are known in the art, and some of the membranes are based on polymeric, ceramic or metal materials well known in the art of membrane science, such as polysulfone, polyimide, polyamide, cellulose acetate, zeolite or palladium. The membrane may have many different shapes and sizes, such as for example in the form of a spiral wound membrane, hollow fibre membrane, tube membrane or plate membrane. The actual selectivity for H₂ for example over propane depends on the material that the membrane is made of, as well as the process conditions, including the temperature and the pressure on the feed side and the permeate side, respectively.

A driving force for transmembrane permeation is provided by a higher pressure on the feed side than on the permeate side. For example, the pressure on the feed side may include a pressure of 1 MPa or higher, such as 2 MPa or higher, or 3 MPa or higher, or 4 MPa or higher, or 5 MPa or higher, and the pressure on the permeate side may include a pressure that is at least 0.1 MPa lower than a pressure on the feed side, such as at least 0.5 MPa lower, or at least 1 MPa lower, or at least 2 MPa lower, or at least 3 MPa lower.

Preferably, the membrane employed in the membrane separation technique is selective for H₂ over propane (permeates most of molecular hydrogen and rejects most of propane). In embodiments wherein the membrane is selective for H₂ over propane, propane rich gas (compared to propane content before membrane separation) is obtained as membrane retentate. The retentate is fed to the thermal cracking optionally after further purification and optionally together with a co-feed. The membrane material and conditions for membrane separation are preferably selected so that the membrane exhibits a selectivity for H₂ over propane of at least 5, such as at least 10, at least 20, at least 30, at least 50, or at least 60, measured as pure component permeability ratio (vol/vol).

If present in the gaseous fraction, CO and hydrocarbons other than propane (methane, ethane, and/or C4+ hydrocarbons) may also be rejected together with propane, while H₂O, CO₂, H₂S and NH₃ may be rejected or partially rejected depending on the membrane type and conditions, e.g. temperature and pressure, of the membrane separation. In otherwords, if present in the gaseous fraction, CO and hydrocarbons other than propane, and possibly H₂O, CO₂, H₂S end up in the retentate with propane.

In certain embodiments, the purification treatment comprises drying. The drying may be performed before or after the H₂ separation. Preferably, the drying is performed after the H₂ separation. The drying may be accomplished using any conventionally known chemical and/or physical method, e.g. using an adsorbent and/or absorbent for water. One particularly preferred embodiment involves drying using molecular sieve dehydration beds.

In certain embodiments, the method of the present disclosure is performed without cryogenic distillation, especially, the purification treatment does not contain or is performed without a cryogenic distillation step.

The present disclosure provides a simple method to produce valuable chemicals from gaseous side streams from hydrotreatment of renewable oxygen containing hydrocarbons without excessive purification steps. The desired composition of the thermal cracking feed, including the desired propane to H₂ ratio (mol-%/mol-%), may be obtained by combining propane and hydrogen containing gaseous fractions from different hydrotreatment effluents and/or by adjusting the optional purification treatment and/or by mixing with a suitable gaseous composition, such as fossil LPG.

In certain embodiments, wherein the hydrotreatment comprises deoxygenation and isomerisation and wherein the deoxygenation and the isomerisation are carried out in separate reactors, separating gaseous fraction from hydrotreatment effluent may be carried out separately for the deoxygenation effluent and the isomerisation effluent. The gaseous fractions of the deoxygenation effluent and the isomerisation effluent may then be combined, optionally after having subjected at least the gaseous fraction of the deoxygenation effluent to purification treatment, which purification treatment preferably comprises at least separation of H₂.

In certain embodiments, wherein separating gaseous fraction from hydrotreatment effluent is carried out separately for the deoxygenation effluent and for the isomerisation effluent, the gaseous fraction of the isomerisation effluent is separated from the liquid fraction of the isomerisation effluent by (fractional) distillation. The gaseous fraction of the isomerisation effluent may be separated as the overhead product of a diesel stabilization process. In certain embodiments, the gaseous fraction of the isomerisation effluent is fed to the thermal cracking without having been subjected to purification treatment, preferably as a co-feed with the optionally purified gaseous fraction of the deoxygenation effluent and optionally a gaseous fossil co-feed.

In certain embodiments, the gaseous fraction of the deoxygenation effluent, optionally having been subjected to purification treatment, is fed to the thermal cracking without co-feeds. In other words, in certain embodiments, the gaseous fraction of the deoxygenation effluent, optionally having been subjected to purification treatment, is the thermal cracking feed. In certain other embodiments, the gaseous fraction of the deoxygenation effluent, optionally having been subjected to purification treatment, is fed to the thermal cracking as co-feed with other hydrocarbons, such as a gaseous fossil co-feed.

Any conventional thermal cracking diluents may be used in the thermal cracking process of the present disclosure. Examples of such thermal cracking diluents comprise steam, molecular nitrogen (N₂), or a mixture thereof. Dilution of the thermal cracking feed lowers the hydrocarbon partial pressure in the thermal cracking coils and favours formation of primary reaction products, such as ethylene and propylene. The dilution also further reduces coke deposition on the thermal cracking coils. Preferably, the thermal cracking is steam cracking, i.e. the thermal cracking diluent is steam.

Preferably, in the context of the present disclosure, the thermal cracking is conducted without the presence of a (solid) catalyst and/or the thermal cracking is steam cracking.

Any conventional thermal cracking additives may be added to the thermal cracking feed of the present disclosure or be co-fed to the thermal cracking furnace with the thermal cracking feed of the present disclosure. Examples of such conventional thermal cracking additives include sulfur containing species (sulfur additives), such as dimethyl disulfide (DMDS), or carbon disulfide (CS₂). DMDS is a particularly preferred sulfur additive. Sulfur additive may be mixed with the thermal cracking feed before feeding the thermal cracking feed to the thermal cracking. Optionally, sulfur additive may be added by injecting into the thermal cracking furnace a diluent, preferably steam, comprising sulfur additive.

Because the thermal cracking feed of the present disclosure already reduces coke formation, it may not be necessary to additize the thermal cracking feed with sulfur, or a low amount of sulfur additive is sufficient. An advantage of a low sulfur content is that cracking products, particularly heavier hydrocarbon fractions, also has a low sulfur content. Typically, heavier hydrocarbon fractions (C5+ hydrocarbons) separated or fractionated from the thermal cracking effluent are not subjected to extensive purification, and therefore sulfur originating from the thermal cracking substantially remains in these fractions. C5-C9 hydrocarbons from the thermal cracking may be used as fuel components. Low or ultra-low sulfur fuels and fuel components are preferred because fuels with a low sulfur content or fuels free from sulfur produce less harmful emissions upon combustion compared to fuels or fuel components with a higher sulfur content.

The thermal cracking of the present disclosure may be carried out at a coil outlet temperature (COT) selected from a wide temperature range. The COT is usually the highest temperature for the thermal cracking feed in the thermal cracker. The thermal cracking may be performed at a COT selected from a range from 750° C. to 920° C. Preferably, the COT is selected from a range from 750° C. to 890° C., further preferably from 820° C. to 880° C., more preferably from 830° C. to 880° C., even more preferably from 850° C. to 880° C. The selectivity towards ethylene is particularly good (the ratio of the wt-% amount of propylene to the wt-% amount of the ethylene in the thermal cracking effluent is particularly low), and the propane conversion and the ethylene yield are particularly high when the COT is selected from the range from 850° C. to 880° C., especially when the COT is 880° C.

The thermal cracking may be carried out at a coil outlet pressure (COP) within a range from 1.3 bar (absolute) to 6 bar (absolute), preferably from 1.3 bar (absolute) to 3 bar (absolute) or/and a flow rate ratio between thermal cracking diluent, preferably steam, and thermal cracking feed (flow rate of diluent [kg/h]/flow rate of thermal cracking feed [kg/h]) within a range from 0.1 to 1, preferably from 0.25 to 0.85.

The thermal cracking process may comprise recycling unconverted reactants, such as propane and/or ethane, back to the thermal cracking furnace. Recycling unconverted reactants increases the overall profitability and the overall yield of the thermal cracking process and/or the overall yield of the desired products ethylene and propylene.

The thermal cracking may be performed in multiple thermal cracking furnaces. The effluents of the multiple thermal cracker furnaces may be combined to form one or more effluent streams optionally transported or conveyed to further processing steps, such as purification and/or fractionation and/or derivatisation and/or polymerisation. Alternatively, the thermal cracking may be performed in a single thermal cracker furnace and the effluent from the single thermal cracking furnace may optionally be transported or conveyed to further processing steps, such as purification and/or fractionation and/or derivatisation and/or polymerisation.

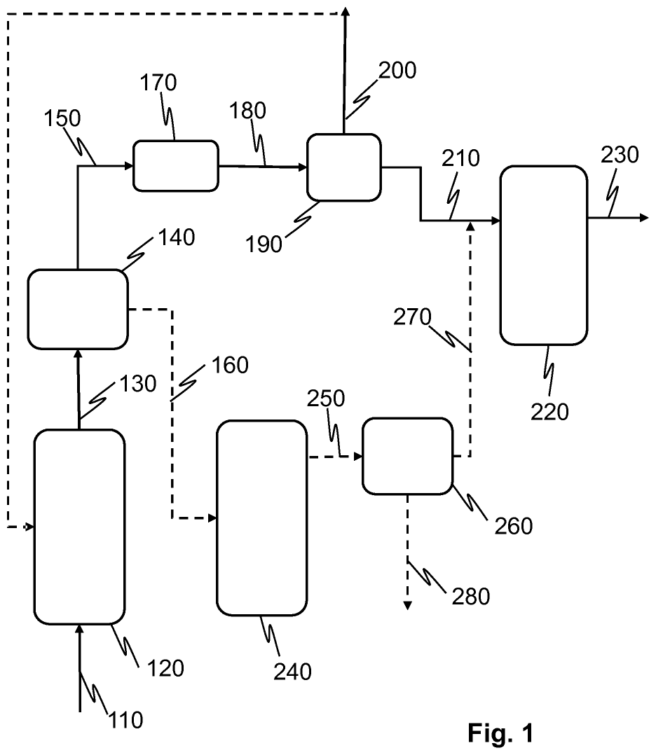

FIG. 1 shows a schematic drawing of an example embodiment of the method of the present disclosure. In FIG. 1, a feed of renewable oxygen containing hydrocarbons 110 is fed to a hydrodeoxygenation reactor 120 wherein the feed of renewable oxygen containing hydrocarbons 110 is subjected to hydrodeoxygenation to produce a hydrodeoxygenation effluent 130. The hydrodeoxygenation effluent 130 is fed to a gas/liquid separator 140 wherein the hydrodeoxygenation effluent is fractionated into a gaseous fraction 150 and a liquid fraction 160. The gaseous fraction 150 of the hydrodeoxygenation effluent is in FIG. 1 fed to an amine absorber 170 wherein the contents of H₂S and CO₂ in the gaseous fraction 150 of the hydrodeoxygenation effluent are decreased, after which the H₂S and CO₂ depleted gaseous fraction 180 of the hydrodeoxygenation effluent is fed to membrane separation 190 to separate therefrom a H₂ stream 200 to decrease the content of H₂ in the H₂S and CO₂ depleted gaseous fraction 180 of the hydrodeoxygenation effluent. Optionally, the H₂ stream 200 is recycled back to the hydrodeoxygenation reactor 120. The H₂, H₂S, and CO₂ depleted gaseous fraction 210 of the hydrodeoxygenation effluent is then fed to a steam cracker 220 wherein it is subjected to steam cracking to obtain a steam cracking effluent 230.

Optionally, in FIG. 1, the liquid fraction 160 of the hydrodeoxygenation effluent is fed to a hydroisomerisation reactor 240 wherein it is subjected to hydroisomerisation to produce a hydroisomerisation effluent 250. The hydroisomerisation effluent 250 is fed to fractionation 260, such as diesel stabilisation, to separate from the hydroisomerisation effluent 250 at least a gaseous fraction 270 and a liquid fraction 280. The gaseous fraction 270 of the hydroisomerisation effluent is then optionally fed as a co-feed with the H₂, H₂S, and CO₂ depleted gaseous fraction 210 of the hydrodeoxygenation effluent to the steam cracker 220 to be subjected to steam cracking.

The present disclosure provides a thermal cracking effluent obtainable with the method of the present disclosure. The thermal cracking effluent of the present disclosure comprises propylene and at least 20 wt-%, preferably at least 25 wt-%, more preferably at least 28 wt-%, even more preferably at least 30 wt-%, such as at least 32 wt-%, ethylene based on the total dry weight of the thermal cracking effluent, in which thermal cracking effluent the ratio of the wt-% amount of propylene to the wt-% amount of ethylene is less than 0.40, preferably less than 0.30, more preferably less than 0.20, such as less than 0.15, and wherein the thermal cracking effluent comprises less than 5.0 wt-%, preferably less than 3.0 wt-%, more preferably less than 2.5 wt-% hydrocarbons having a carbon number of at least C5 based on the total dry weight of the thermal cracking effluent.

In certain embodiments, the thermal cracking effluent comprises less than 50 wt-% ethylene, such as less than 45 wt-%, or less than 40 wt-% ethylene based on the total dry weight of the thermal cracking effluent.

In certain embodiments, the ratio of the wt-% amount of propylene to the wt-% amount of ethylene in the thermal cracking effluent is at least 0.05, at least 0.075 or at least 0.10.

In certain embodiments, the thermal cracking effluent comprises more than 0.5 wt-%, preferably more than 1.0 wt-% hydrocarbons having a carbon number of at least C5 based on the total dry weight of the thermal cracking effluent.

In certain embodiments, the sum of the wt-% amounts of benzene, toluene, and xylene (BTX) in the thermal cracking effluent is less than 2.0 wt-%, preferably less than 1.5 wt-% based on the total dry weight of the thermal cracking effluent.

In certain embodiments, the thermal cracking effluent comprises butadiene less than 2.5 wt-%, preferably less than 2.0 wt-% based on the total dry weight of the thermal cracking effluent.

In certain embodiments, the sum of the wt-% amounts of methyl acetylene and propadiene (MAPD) in the thermal cracking effluent is less than 0.3 wt-% of based on the total dry weight of the thermal cracking effluent.

In certain preferred embodiments, the thermal cracking effluent comprises propylene and at least 20 wt-% and less than 50 wt-% ethylene, more than 0.5 wt-% and less than 5.0 wt-% hydrocarbons having a carbon number of at least C5, and less than 2.5 wt-% butadiene based on the total dry weight of the thermal cracking effluent, in which thermal cracking effluent the ratio of the wt-% amount of propylene to the wt-% amount of ethylene is at least 0.05 and less than 0.40, and the sum of the wt-% amounts of benzene, toluene, and xylene (BTX) in the thermal cracking effluent is less than 2.0 wt-%, and the sum of the wt-% amounts of methyl acetylene and propadiene (MAPD) in the thermal cracking effluent is less than 0.3 wt-% based on the total dry weight of the thermal cracking effluent.

The thermal cracking effluent may be subjected to purification and/or fractionation. Any conventional purification and/or fractionation methods may be employed.

In certain embodiments, the method comprises fractionating the thermal cracking effluent. The fractionation may comprise separating from the thermal cracking effluent a C2 fraction (ethylene fraction), C3 fraction (propylene fraction), and/or a C4 fraction. Further, a C5-C9 (PyGas) fraction and/or a C10+(PFO) fraction may be separated. In certain embodiments, at least a C2 fraction and a C3 fraction are separated from the cracking effluent.

The C2 fraction (ethylene fraction) and the C3 fraction (propylene fraction) may be respectively used to produce polymers, optionally after having been subjected to purification treatment and/or derivatisation. Thus, in certain embodiments, the method comprises separating from the thermal cracking effluent a C2 fraction, a C3 fraction, or both, and subjecting the C2 fraction, the C3 fraction, or both to polymerisation treatment optionally in the presence of copolymerisable monomer(s) and/or additive(s). At least a portion of the C2 fraction, C3 fraction, or both may be subjected to purification treatment and/or at least partially derivatized before being subjected to the polymerisation treatment optionally in the presence of copolymerisable monomer(s) and/or additive(s).

The purification may be conducted e.g. by any known purification technique such as distillation, extraction, selective hydrotreatment to remove MAPD, etc. The purification treatment increases the ethylene or propylene content of the C2 or C3 fraction, respectively, and/or removes impurities/contaminants from the respective fraction.

Optionally, at least a portion of the hydrocarbons included in the thermal cracking effluent may be further processed into a derivative or derivatives of the respective compound. The derivatizing may be conducted e.g. by any known chemical modification technique providing monomers e.g. with anionically and/or cationically charged group(s), hydrophobic group(s), or any other desired characteristic.

### Examples

The following examples are provided to better illustrate the claimed invention and are not to be interpreted as limiting the scope of the invention. To the extent that specific materials are mentioned, it is merely for purposes of illustration and is not intended to limit the invention.

Steam cracking simulations were carried out using COILSIM1D.

Four different feeds were simulated: a 100 wt-% propane feed (F1), a renewable propane composition comprising 95.9 wt-% propane and no molecular hydrogen (F2), a renewable propane composition comprising 66 wt-% propane and 9.1 wt-% molecular hydrogen (F3), and a renewable propane composition comprising 66 wt-% propane, but wherein the molecular hydrogen had been replaced with molecular nitrogen (F4). The simulated feeds are described in Table 1. The weight percentages shown in Table 1 are based on the dry composition of each feed, i.e. possible H₂O content has been excluded.

TABLE 1
Compositions of feeds F1, F2, F3, and F4.

| Component | Unit | F1 | F2 | F3 | F4 |
|---|---|---|---|---|---|
| H₂ | wt-% | | | 9.1 | |
| N₂ | wt-% | | | 0.9 | 10 |
| CO | wt-% | | | 2.5 | 2.5 |
| CO₂ | wt-% | | | 0.3 | 0.3 |
| CH₄ | wt-% | | | 5.8 | 5.8 |
| C₂H₆ | wt-% | | 0.6 | 3.8 | 3.8 |
| C₃H₈ | wt-% | 100 | 95.9 | 66 | 66 |
| isoC₄H₁₀ | wt-% | | 1.3 | 2.2 | 2.2 |
| nC₄H₁₀ | wt-% | | 1.6 | 3 | 3 |
| isoC5 | wt-% | | | 1.5 | 1.5 |
| nC5 | wt-% | | 0.6 | 1.5 | 1.5 |
| isoC6 | wt-% | | | 1.7 | 1.7 |
| nC6 | wt-% | | | 1.7 | 1.7 |
| Total C2+ | wt-% | 100 | 100 | 81.4 | 81.4 |
| hydrocarbons | | | | | |

In Table 1, iso refers to branched molecules and n to normal or unbranched molecules.

Table 2 shows calculated conversions of the feed compositions (wt-% values of Table 1) to mol-%. The mole percentages shown in Table 2 are based on the dry composition of each feed.

TABLE 2
Calculated conversions of the feed compositions
(wt-% values of Table 1) to mole percentages.

| Component | Unit | F1 | F2 | F3 | F4 |
|---|---|---|---|---|---|
| H₂ | mol-% | | | 66.4 | |
| N₂ | mol-% | | | 0.5 | 13.7 |
| CO | mol-% | | | 1.3 | 3.4 |
| CO₂ | mol-% | | | 0.1 | 0.3 |
| CH₄ | mol-% | | | 5.3 | 13.9 |
| C₂H₆ | mol-% | | 0.9 | 1.9 | 4.8 |
| C₃H₈ | mol-% | 100 | 96.5 | 22 | 57.4 |
| isoC₄H₁₀ | mol-% | | 1 | 0.6 | 1.5 |
| nC₄H₁₀ | mol-% | | 1.2 | 0.8 | 2 |
| isoC5 | mol-% | | | 0.3 | 0.8 |
| nC5 | mol-% | | 0.4 | 0.3 | 0.8 |
| isoC6 | mol-% | | | 0.3 | 0.8 |
| nC6 | mol-% | | | 0.3 | 0.8 |
| Total C2+ | mol-% | 100 | 100 | 26.4 | 68.8 |
| hydrocarbons | | | | | |

In Table 2, iso refers to branched molecules and n to normal or unbranched molecules.

The simulations were performed using the feed compositions defined in wt-% values, i.e. the feed compositions of Table 1. Steam cracking of each feed was simulated at three different coil outlet temperatures (COT): 830° C., 850° C., and 880° C. The coil outlet pressure (COP) was kept at 2.093 atm (about 2.12 bar (absolute)), the coil inlet temperature was kept at 645° C., the feed flow was kept at 625 kg/h, and the steam dilution was kept at 0.4 (kg/h steam to kg/h feed) in the simulations. Steam cracking effluents obtained in the simulations are shown in Table 3 (F1 and F2) and Table 4 (F3 and F4).

TABLE 3
Simulated steam cracking effluents of feeds F1 and F2, respectively.

| Feed | F1 | | | F2 | | |
|---|---|---|---|---|---|---|
| COT, ° C. | 830 | 850 | 880 | 830 | 850 | 880 |
| Total C1-C4 | 95.4 | 93.3 | 89.1 | 95.3 | 93.1 | 88.8 |
| hydrocarbons, wt-% | | | | | | |
| Total C5-C9 | 3.2 | 5.1 | 8.8 | 3.4 | 5.3 | 9.0 |
| hydrocarbons, wt-% | | | | | | |
| Total C10+ | 0.04 | 0.11 | 0.49 | 0.04 | 0.12 | 0.51 |
| hydrocarbons, wt-% | | | | | | |
| Propylene/Ethylene ratio | 0.69 | 0.53 | 0.31 | 0.69 | 0.53 | 0.31 |
| (wt-%/wt-%) | | | | | | |
| Propane conversion [%] | 71.8 | 82.2 | 93.4 | 72.2 | 82.5 | 93.5 |
| Coking rate [mm/month] | 8.8 | 17.2 | 40.7 | 8.9 | 17.3 | 40.4 |
| Ethylene, wt-% | 26.5 | 31.5 | 37.3 | 26.4 | 31.5 | 37.1 |
| Propylene, wt-% | 18.3 | 16.7 | 11.7 | 18.3 | 16.7 | 11.7 |
| 1,3-Butadiene, wt-% | 1.4 | 2.0 | 2.7 | 1.4 | 2.0 | 2.7 |
| H₂, wt-% | 1.3 | 1.5 | 1.7 | 1.3 | 1.5 | 1.7 |
| CO, wt-% | 0 | 0 | 0 | 0 | 0 | 0 |
| CO₂, wt-% | 0 | 0 | 0 | 0 | 0 | 0 |
| Methane, wt-% | 16.6 | 20.1 | 24.9 | 16.7 | 20.2 | 25.0 |
| Acetylene, wt-% | 0.20 | 0.40 | 0.86 | 0.22 | 0.41 | 0.87 |
| Ethane, wt-% | 2.6 | 3.1 | 3.4 | 2.7 | 3.1 | 3.4 |
| Methylacetylene (MA), wt-% | 0.2 | 0.3 | 0.4 | 0.2 | 0.3 | 0.4 |
| Propadiene, (PD), wt-% | 0.1 | 0.1 | 0.1 | 0.1 | 0.1 | 0.1 |
| Total MAPD, wt-% | 0.3 | 0.4 | 0.5 | 0.3 | 0.4 | 0.5 |
| Propane, wt-% | 28.2 | 17.8 | 6.6 | 27.0 | 17.0 | 6.3 |
| Isoprene, wt-% | 0.40 | 0.61 | 0.87 | 0.46 | 0.67 | 0.91 |
| CPD, wt-% | 0.25 | 0.44 | 0.75 | 0.26 | 0.45 | 0.76 |
| Benzene, wt-% | 0.85 | 1.63 | 3.80 | 0.88 | 1.70 | 3.90 |
| Toluene, wt-% | 0.14 | 0.32 | 0.68 | 0.15 | 0.34 | 0.70 |
| Xylene, wt-% | 0.02 | 0.05 | 0.13 | 0.02 | 0.06 | 0.14 |
| C4H4, wt-% | 0.16 | 0.22 | 0.25 | 0.17 | 0.22 | 0.25 |
| Total products, wt-% | 100 | 100 | 100 | 100 | 100 | 100 |

TABLE 4
Simulated steam cracking effluents of feeds F3 and F4, respectively.

| Feed | F3 | | | F4 | | |
|---|---|---|---|---|---|---|
| COT, ° C. | 830 | 850 | 880 | 830 | 850 | 880 |
| Total C1-C4 | 86.5 | 86.18 | 85.55 | 89.11 | 87.35 | 84.73 |
| hydrocarbons, wt-% | | | | | | |
| Total C5-C9 | 1.3 | 1.7 | 2.3 | 5.95 | 7.04 | 8.29 |
| hydrocarbons, wt-% | | | | | | |
| Total C10+ | 0.02 | 0.05 | 0.14 | 0.7 | 1.3 | 2.6 |
| hydrocarbons, wt-% | | | | | | |
| Propylene/Ethylene ratio | 0.37 | 0.26 | 0.13 | 0.34 | 0.26 | 0.16 |
| (wt-%/wt-%) | | | | | | |
| Propane conversion [%] | 85.7 | 91.7 | 97.3 | 92.3 | 95.8 | 98.7 |
| Coking rate [mm/month] | 2.9 | 4.3 | 6.9 | 20.3 | 28.1 | 42.5 |
| Ethylene, wt-% | 28.2 | 31.3 | 34.7 | 30.3 | 31.5 | 31.9 |
| Propylene, wt-% | 10.3 | 8.1 | 4.6 | 10.2 | 8.0 | 5.0 |
| 1,3-Butadiene, wt-% | 1.0 | 1.2 | 1.4 | 2.2 | 2.4 | 2.5 |
| H₂, wt-% | 9.4 | 9.3 | 9.2 | 1.4 | 1.5 | 1.6 |
| CO, wt-% | 2.5 | 2.5 | 2.5 | 2.5 | 2.5 | 2.5 |
| CO₂, wt-% | 0.3 | 0.3 | 0.3 | 0.3 | 0.3 | 0.3 |
| Methane, wt-% | 26.0 | 28.7 | 32.2 | 25.1 | 26.8 | 29.0 |
| Acetylene, wt-% | 0.31 | 0.46 | 0.78 | 0.54 | 0.74 | 1.18 |
| Ethane, wt-% | 9.2 | 9.5 | 9.3 | 4.0 | 3.7 | 3.2 |
| Methylacetylene (MA), wt-% | 0.1 | 0.1 | 0.1 | 0.3 | 0.3 | 0.3 |
| Propadiene (PD), wt-% | 0.1 | 0.1 | 0.1 | 0.1 | 0.1 | 0.1 |
| Total MAPD, wt-% | 0.2 | 0.2 | 0.2 | 0.4 | 0.4 | 0.4 |
| Propane, wt-% | 9.5 | 5.5 | 1.8 | 5.1 | 2.8 | 0.8 |
| Isoprene, wt-% | 0.06 | 0.06 | 0.05 | 0.26 | 0.25 | 0.20 |
| CPD, wt-% | 0.32 | 0.44 | 0.60 | 1.11 | 1.24 | 1.27 |
| Benzene, wt-% | 0.42 | 0.63 | 1.02 | 2.35 | 2.93 | 3.62 |
| Toluene, wt-% | 0.05 | 0.07 | 0.11 | 0.53 | 0.69 | 0.91 |
| Xylene, wt-% | 0.01 | 0.01 | 0.02 | 0.10 | 0.15 | 0.21 |
| C4H4, wt-% | 0.02 | 0.05 | 0.11 | 0.10 | 0.16 | 0.34 |
| Total products, wt-% | 100 | 100 | 100 | 100 | 100 | 100 |

C10+ refers to compounds having a carbon number of at least C10 (C10 and higher). The propane conversion values are obtained from the simulations. Relatively good approximation of propane conversion could also be obtained by calculating as follows: 100%×((wt-% amount of propane in thermal cracking feed—wt-% amount of propane in thermal cracking effluent)/(wt-% amount of propane in thermal cracking feed)). The propylene to ethylene ratio in Tables 3 and 4 is given as the ratio of the wt-% amount of propylene to the wt-% amount of ethylene in the steam cracking effluent. C1-C5 hydrocarbons may be referred to as pyrolysis gasoline (Pygas) and hydrocarbons having a carbon number of at least C10 may be referred to as pyrolysis fuel oil (PFO). The weight percentages in Tables 3 and 4 are calculated based on the dry weight of the respective thermal cracking effluent, i.e. excluding possible H₂O content. As seen in Tables 3 and 4, surprisingly steam cracking of feeds F3 and F4 containing less propane (66 wt-%) than feeds F1 and F2 (100 wt-% propane and 95.9 wt-% propane, respectively) achieved very high propane conversions compared to feeds F1 and F2. The propane conversion increased when the COT was increased from 830° C. to 850° C., and from 850° C. to 880° C.

Also, the ethylene yields of feeds F3 and F4 with the lower propane content were substantially higher than what was expected based on the hydrocarbon composition of said feeds. The total yields of C2 compounds of feeds F3 and F4 were also higher than excepted based on the amount of C2+ hydrocarbons in said feeds. In feeds F3 and F4, the amount of C2+ hydrocarbons was 86.3 wt % when it in feeds F1 and F2 was 100 wt-%. That is, in feeds F3 and F4 the wt-% of compounds that can convert to ethylene and/or propylene in the steam cracking was notably lower than in feeds F1 and F2.

Additionally, feeds F3 and F4 had improved selectivity towards ethylene (lower ratio of the wt-% amount propylene to the wt-% amount of ethylene in the steam cracking effluent) compared to the high propane feeds F1 and F2. At higher COT temperatures, e.g. 880° C., the presence of H₂ in feed F3 enhanced selectivity towards ethylene even further compared to F4 without H₂ content.

The ratios of the wt-% amount of propylene to the total wt-% amount of C3 compounds in the steam cracking effluents were higher for feeds F3 and F4 compared to the high propane feeds F1 and F2. A higher ratio of propylene to total C3 compounds facilitates and lowers energy consumption of purification of propylene from the close-boiling C3 compounds, particularly propane. Feeds F3 and F4 also yielded less pyrolysis gasoline (C5-C9) than feeds F1 and F2.

Comparison of the steam cracking effluents and coking rates of F3 and F4 show that feed F3 has beneficial effects not obtained by F4. Accordingly, the beneficial effects of F3 are not obtained due to lower propane content compared to feeds F1 and F2 alone, but the presence of molecular hydrogen in feed F3 is also important. Based on the results of Tables 3 and 4, it is concluded that even though mere dilution of the propane content did improve propane conversion while providing an unexpectedly good ethylene yield, for the other benefits and especially for reducing the coke formation presence of H₂ in the steam cracking feed was required.

As seen in Tables 3 and 4, surprisingly, feed F3 had a significantly lower coking rate compared to each of feeds F1, F2, and F4. Also, feed F3 had lower yields of butadiene, MAPD contaminants (methyl acetylene and propadiene), aromatic compounds, especially BTX compounds (benzene, toiluene, xylene), and C10+ compounds compared to the other feeds F1, F2, and F4. The lower yield of aromatic compounds is, without being bound to any theory, believed to contribute to the lower coking rate of F3. MAPD contaminants are highly reactive and therefore undesired in the steam cracking effluent.

It can be seen in Table 4 that surprisingly, the compositions of the steam cracking effluents and coking rates of F4, without H₂ content but otherwise similar to F3, are substantially different compared to the compositions of the steam cracking effluents and coking rates of F3 comprising 9.1 wt-% H₂. Especially, the amount of C10+ compounds in the steam cracking effluents becomes quite high when there is no H₂ content in the feed. The coke formation of F4 is more in a range with the high propane feeds F1 and F2, and no reduction in the coking rate is seen for F4. Also, the yields of MAPD contaminants (mixture of methyl acetylene and propadiene) were lower in the presence of H₂ (compared to presence N₂ in F4). It can thus be concluded that the presence of H₂ in F3 results in less butadiene, less BTX, less MAPD, and less pyrolysis fuel oil (C10+ hydrocarbons) in the steam cracking effluent and a lower coking rate compared to the otherwise similar composition F4 where the H₂ has been replaced with N₂.

Specific yields per C2+ hydrocarbons (specific yields per valuable hydrocarbons) were calculated for certain thermal cracking products. The calculated specific yields per valuable hydrocarbons are shown in Table 5.

TABLE 5
Specific yields per C2+ hydrocarbons (specific yields
per valuable hydrocarbons) of certain thermal cracking products.

| Feedstock | F1 | | | F2 | | | F3 | | | F4 | | |
|---|---|---|---|---|---|---|---|---|---|---|---|---|
| COT [° C.] | 830 | 850 | 880 | 830 | 850 | 880 | 830 | 850 | 880 | 830 | 850 | 880 |
| Ethylene, wt-% | 27 | 31.5 | 37 | 26 | 32 | 37 | 28 | 31 | 35 | 30 | 32 | 32 |
| Acetylene, wt-% | 0.2 | 0.4 | 0.9 | 0.2 | 0.4 | 0.9 | 0.4 | 0.6 | 1 | 0.7 | 0.9 | 1.4 |
| Ethane, wt-% | 2.6 | 3.1 | 3.4 | 2.7 | 3.1 | 3.4 | 11 | 12 | 11 | 4.9 | 4.6 | 3.9 |
| Total C2, wt-% | 29 | 35 | 42 | 29 | 35 | 41 | 49 | 54 | 58 | 44 | 45 | 46 |
| Propylene, wt-% | 18 | 16.7 | 12 | 18 | 17 | 12 | 10 | 8.1 | 4.6 | 10 | 8 | 5 |
| MAPD, wt-% | 0.3 | 0.4 | 0.5 | 0.3 | 0.4 | 0.5 | 0.2 | 0.3 | 0.2 | 0.5 | 0.5 | 0.5 |
| Propane, wt-% | 28 | 17.8 | 6.6 | 27 | 17 | 6.3 | 12 | 6.8 | 2.2 | 6.3 | 3.4 | 1 |
| Total C3, wt-% | 47 | 34.8 | 19 | 46 | 34 | 19 | 22 | 15 | 7.1 | 17 | 12 | 6.5 |
| Total C2 + C3, wt-% | 76 | 69.8 | 60 | 75 | 69 | 60 | 71 | 69 | 65 | 61 | 57 | 52 |
| BTX, wt-% | 1 | 2 | 4.6 | 1.1 | 2.1 | 4.7 | 0.6 | 0.9 | 1.4 | 3.7 | 4.6 | 5.8 |
| PyGas (C5-C9), wt-% | 3.2 | 5.1 | 8.8 | 3.4 | 5.3 | 9 | 1.3 | 1.7 | 2.3 | 5.8 | 6.9 | 8.2 |
| PFO (C10+), wt-% | <0.1 | 0.1 | 0.5 | <0.1 | 0.1 | 0.5 | <0.1 | 0.1 | 0.1 | 1 | 1.5 | 2.8 |
| Coke formation | 8.8 | 17.2 | 41 | 8.9 | 17 | 40 | 2.9 | 4.3 | 6.9 | 20 | 28 | 43 |
| [mm/month] | | | | | | | | | | | | |

The results shown in Table 5 confirm the observation that the presence of molecular hydrogen in the thermal cracking feed significantly reduces the yields of products heavier than C3. In turn ethylene yield is slightly enhanced. Furthermore, the presence of molecular hydrogen in the thermal cracking feed significantly reduces coke formation. F3 also has a high specific yield per valuable hydrocarbons of C2 compounds, such as ethane.

It may thus be concluded that H₂ has an impact on the chemistry during steam cracking and does thus not function in the present context as a mere diluent. The benefits obtained by F3 but not F4 are, without being bound to any theory, believed to be due to H₂ controlling formation and/or further reactions of reactive species like unsaturated compounds, although the underlying mechanism is not known. H₂ in the steam cracking feed seems to convert a lot of unsaturated hydrocarbons preventing the formation of secondary reactions and therefore heavy products. It is also surprising that despite of the presence of H₂ in F3, especially ethylene, and also propylene, are obtained with good yields, i.e. the presence of H₂ did not lead to saturation of the double bonds of said compounds.

From the results in Tables 3 and 4, it can be seen that for the feeds without H₂ content (F1, F2, F4), whether with high or low propane content, H₂ is generated (from at least 1.3 wt-% to up to 1.74 wt-%) during steam cracking of these feeds. However, apparently the H₂ formed during steam cracking alone is not sufficient for obtaining the benefits of having H₂ present in the feed already from the beginning of the steam cracking. Although the underlying mechanism is not known and without being bound to any theory, it is possible that the early presence of H₂ prevents or reduces formation of highly reactive species, or quenches them, thereby controlling the chain of subsequent reactions.

Surprisingly, feed F3 has clear benefits both over the high propane feeds F1 and F2 as well as F4 without H₂ content. Surprisingly, by decreasing the propane content in the feed from the very high propane contents of feeds F1 and F2 and including molecular hydrogen in the steam cracking feed a beneficial steam cracking chemistry and low coking rate are achieved. Increasing the COT from 830° C. to 850° C. and from 850° C. to 880° C. further promotes formation of ethylene, increases selectivity towards ethylene as well as increases propane conversion.

Various embodiments have been presented. It should be appreciated that in this document, words comprise, include, and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1.-24. (canceled)

25. A method comprising:
providing a thermal cracking feed containing molecular hydrogen (H2) and from 10 mol-% to 60 mol-% propane based on a total dry amount of substance of the thermal cracking feed, in which thermal cracking feed a ratio of the mol-% amount of propane to the mol-% amount of molecular hydrogen is within a range from 0.10 to 2.5; and
subjecting the thermal cracking feed to thermal cracking to obtain a thermal cracking effluent including ethylene.

26. The method according to claim 25, wherein the thermal cracking feed comprises:
from 15 mol-% to 50 mol propane based on the total dry amount of substance of the thermal cracking feed, and/or the ratio of the mol-% amount of propane to the mol-% amount of molecular hydrogen in the thermal cracking feed is within a range from 0.10 to 2.2.

27. The method according to claim 25, wherein a ratio of a mol-% amount of hydrocarbons having a carbon number of at least C2 to the mol-% amount of molecular hydrogen in the thermal cracking feed is within a range from 0.10 to 2.5.

28. The method according to claim 25, wherein the thermal cracking feed comprises:
from 1 mol-% to 8 mol-% hydrocarbons having a carbon number of at least C4 based on the total dry amount of substance of the thermal cracking feed.

29. The method according to claim 25, wherein the thermal cracking feed comprises:
from 0 mol-% to 10 mol-% ethane based on the total dry amount of substance of the thermal cracking feed.

30. The method according to claim 25, wherein the thermal cracking feed comprises:
from 5 mol-% to 80 mol-%, and/or from 10 mol-% to 80 mol-% molecular hydrogen based on the total dry amount of substance of the thermal cracking feed.

31. The method according to claim 25, wherein a sum of mol-% amounts of methane, CO, CO2, NH3 and H2S in the thermal cracking feed is within a range from 0 mol-% to 15 mol-% and/or from 0.1 mol-% to 15 mol-% based on the total dry amount of substance of the thermal cracking feed.

32. The method according to claim 25, wherein a biogenic carbon content of the thermal cracking feed is at least 50 wt-% based on a total weight of carbon (TC) of the thermal cracking feed (EN 16640 (2017)).

33. The method according to claim 25, wherein the thermal cracking is steam cracking.

34. The method according to claim 25, comprising:
conducting the thermal cracking at a coil outlet temperature (COT) within a range from 750° C. to 920° C. and/or at a coil outlet pressure (COP) within a range from 1.3 bar (absolute) to 6 bar (absolute) and/or at a flow rate ratio between thermal cracking diluent and the thermal cracking feed (flow rate of diluent [kg/h]/flow rate of thermal cracking feed [kg/h]) within a range from 0.1 to 1.

35. The method according to claim 25, wherein providing a thermal cracking feed comprises:
subjecting renewable oxygen containing hydrocarbons to hydrotreatment comprising deoxygenation to obtain hydrotreatment effluent;
separating a gaseous fraction from the hydrotreatment effluent; and
providing the gaseous fraction or its mixture with a gaseous fossil co-feed as the thermal cracking feed.

36. The method according to claim 25, comprising:
separating an ethylene fraction from the thermal cracking effluent;
subjecting the ethylene fraction to polymerisation treatment to produce polymers; and/or
separating a propylene fraction from the thermal cracking effluent, and subjecting the propylene fraction to polymerisation treatment to produce polymers.

37. A thermal cracking feed comprising:
molecular hydrogen (H2); and
from 10 mol-% to 60 mol-% propane based on a total dry amount of substance of the thermal cracking feed, in which thermal cracking feed a ratio of the mol-% amount of propane to a mol-% amount of molecular hydrogen is within a range from 0.10 to 2.5 and a ratio of a mol-% amount of hydrocarbons having a carbon number of at least C2 to a mol-% amount of molecular hydrogen is within a range from 0.10 to 2.5.

38. The thermal cracking feed according to claim 37, comprising:
from 15 mol-% to 50 mol-%, and/or from 20 mol-% to 45 mol-% propane based on the total dry amount of substance of the thermal cracking feed; and/or
wherein the ratio of the mol-% amount of propane to the mol-% amount of molecular hydrogen is within a range from 0.10 to 2.2, and/or from 0.18 to 2.2.

39. The thermal cracking feed according to claim 37, comprising:
from 1 mol-% to 8 mol-% hydrocarbons having a carbon number of at least C4 based on the total dry amount of substance of the thermal cracking feed.

40. The thermal cracking feed according to claim 37, comprising:
from 0 mol-% to 10 mol-% ethane based on the total dry amount of substance of the thermal cracking feed.

41. The thermal cracking feed according to claim 37, comprising:
from 5 mol-% to 80 mol-% molecular hydrogen based on the total dry amount of substance of the thermal cracking feed.

42. The thermal cracking feed according to claim 37, wherein a sum of mol-% amounts of methane, CO, CO2, NH3 and H2S in the thermal cracking feed is within a range from 0 mol-% to 15 mol-% or from 0.1 mol-% to 15 mol-% based on the total dry amount of substance of the thermal cracking feed.

43. The thermal cracking feed according to claim 37, wherein a biogenic carbon content of the thermal cracking fees is at least 50 wt-% based on a total weight of carbon (TC) of the thermal cracking feed (EN 16640 (2017)).

44. A thermal cracking effluent comprising:
propylene; and
at least 20 wt-% ethylene based on a total dry weight of the thermal cracking effluent, in which thermal cracking effluent a ratio of a wt-% amount of propylene to a wt-% amount of ethylene is less than 0.40 and wherein the thermal cracking effluent includes less than 5.0 wt-% hydrocarbons having a carbon number of at least C5 based on the total dry weight of the thermal cracking effluent.
